(19) United States
(12) Patent Application Publication
(10) Pub. No.: US 2007/0088388 A1
(43) Pub. Date: Apr. 19, 2007

(54) Delivery device for implant with dual attachment sites

(76) Inventors: Steven W. Opolski, Carlisle, MA (US); David J. Callaghan, Boston, MA (US)

Correspondence Address: Kirkpatrick & Lockhart Preston Gates Ellis LLP, STATE STREET FINANCIAL CENTER, One Lincoln Street, BOSTON, MA, 02111-2950, US

(21) Appl. No.: 11/516,305

(22) Filed: Sep. 6, 2006

Related U.S. Application Data
(60) Provisional application No. 60/718,518, filed on Sep. 19, 2005.

(51) Int. Cl. A61B 17/08

(52) U.S. Cl. 606/213

(57) ABSTRACT

The invention generally relates to systems and methods for percutaneous closure of intra-cardiac openings, such as a patent foramen ovale (PFO). In one embodiment, a delivery system includes a first attachment mechanism and a second attachment mechanism attached to a closure device for implantation in an intra-cardiac opening. The delivery system can be used to deliver a closure device to the intra-cardiac opening, or to retrieve or re-position a closure device within the intra-cardiac opening.

### RELATED APPLICATION DATA

This application incorporates by reference, and claims priority to and the benefit of, U.S. Provisional Patent Application No. 60/718,518, filed on Sep. 19, 2005.

### TECHNICAL FIELD

The invention generally relates to devices, systems, and related methods for closing intracardiac openings. More particularly, the invention features devices, systems, and related methods for the percutaneous transluminal closure of a patent foramen ovale (PFO) and other intracardiac defects.

### BACKGROUND

The human heart is divided into four compartments or chambers. The left and right atria are located in the upper portion of the heart and the left and right ventricles are located in the lower portion of the heart. The left and right atria are separated from each other by a muscular wall, the interatrial septum, and the ventricles are separated by the interventricular septum.

Either congenitally or by acquisition, abnormal openings (holes or shunts) can occur between the chambers of the heart or between the great vessels, causing inappropriate blood flow. Such deformities are usually congenital and originate during fetal life when the heart forms from a folded tube into a four chambered, two-unit, i.e., atrial and ventricular, system. The septal deformities result from the incomplete formation of the septum, or muscular wall, between the left and right chambers of the heart and can cause significant problems.

One such septal deformity or defect, a patent foramen ovale, is a persistent tunnel with a flap-like opening in the wall between the right atrium and the left atrium of the heart. Since left atrial pressure is normally higher than right atrial pressure, the flap typically stays closed. Under certain conditions, however, right atrial pressure exceeds left atrial pressure, creating the possibility for right to left shunting of venous blood that can allow blood clots and other toxins to enter the systemic circulation. This is particularly problematic for patients who have deep vein thrombosis or clotting abnormalities.

Nonsurgical (i.e., percutaneous) closure of a patent foramen ovale and similar cardiac openings, such as an atrial septal defect or a ventricular septal defect, can be achieved using a variety of mechanical closure devices. These closure devices typically have a structural framework with or without a scaffold material attached thereto.

Currently available delivery devices, however, are often difficult to operate. Current delivery devices are stiff, making it difficult to accurately position the device. Therefore, they do not allow sufficient operator flexibility, and do not allow re-positioning, re-deployment or retrieval of the device in case of procedural complications. Moreover, when current delivery devices are released from the delivered implant, the tension or torque created by positioning the rigid delivery device within the confines of the anatomical location causes the delivery device to spring away with significant force, potentially displacing the implant or injuring the patient. Furthermore, current delivery devices cannot reconnect to the displaced implant for readjustment or recapture.

Improved devices, systems, and related methods for closing cardiac openings, such as, for example, a patent foramen ovale, are, therefore, needed.

### SUMMARY OF THE INVENTION

A delivery system of the present invention allows a physician to percutaneously place a medical implant within a patient with greater control and accuracy via a percutaneous transluminal route, e.g., the femoral vein. The delivery system includes a first attachment mechanism and a second attachment mechanism, both attachable to the medical implant. Each of the first and second attachment mechanisms is independently releasable and controllable, such that the physician is able to release the medical implant in sequential stages. Additionally, the present invention allows an operator, e.g., a physician to recapture the medical implant after release of either or both of the attachment mechanisms to reposition or remove the implant from the patient.

In one aspect, the invention is a delivery system for delivering a medical implant to an anatomical site in a patient. The delivery system comprises a first attachment mechanism sized and shaped for insertion into a mammalian blood vessel. The first attachment mechanism comprises a first locking arm having a shaped locking portion at a distal end. The first attachment mechanism defines a lumen and comprises a first locking member at a distal end.

The delivery system further comprises a second attachment mechanism. The second attachment mechanism is slideably receivable in the lumen of the first attachment mechanism and comprises a second locking member at a distal end. The first attachment mechanism and the second attachment mechanism hold the medical implant for delivery to an anatomical site in a patient.

According to one embodiment, the delivery system further comprises a lumen and a delivery catheter slideably disposed over the first attachment mechanism. The delivery catheter allows the first locking member to transition between a first position and a second position, by immobilizing the first locking member in the first position when the delivery catheter is slid over the first locking member and releasing the first locking member into the second position when the delivery catheter is slid away from the first locking member.

According to one embodiment, the first attachment mechanism reversibly transitions between a first position and a second position. According to another embodiment, the first locking member transitions between a first position, wherein the first locking member is substantially parallel to a longitudinal axis of the first attachment mechanism, and a second position, wherein the first locking member is angled outward from a longitudinal axis of the first attachment mechanism.

According to one embodiment, the first attachment mechanism comprises a male locking member and a female lock receiving member. According to this embodiment, the male locking member comprises the first locking member. According to another embodiment, the male locking member further comprises a guide finger. In another embodiment, the first attachment mechanism comprises a tubular body co-extensive with the guide finger.

According to one embodiment, the first locking member comprises an elongate locking arm. In another embodiment, the elongate locking arm comprises a locking portion at a free end of the elongate locking arm. According to one embodiment, the locking portion comprises a shape selected from the group consisting of a circle, oval, ellipse, rectangle, square, hook, triangle, L, and T. According to another embodiment, the first attachment mechanism comprises a plurality of first locking members spaced a substantially equal distance apart around the circumference of the first attachment mechanism.

According to one embodiment, the second attachment mechanism is slideably disposed within a lumen of the first attachment mechanism. According to various other embodiments of the invention, the second attachment mechanism comprises a threaded attachment member, a ball and socket attachment member, a tensioned clamp and ball attachment member, a collet and ball attachment member, a magnetic attachment member or a releasable suture. According to another embodiment, the first attachment mechanism locks and releases independent of the second attachment mechanism.

According to one embodiment, the delivery system further comprises at least one flexible section. According to another embodiment, the delivery system further comprises a delivery sheath.

In another aspect, the invention is a method for delivering a septal occluder to an intracardiac location in a patient. The method comprises positioning a delivery system comprising a first attachment mechanism and a second attachment mechanism for delivering a medical implant. The first attachment mechanism comprises a first locking member and the first locking member comprises a locking arm having a shaped locking portion. The second attachment mechanism comprises a second locking body. The method comprises positioning the septal occluder at the anatomical location, transitioning the first attachment mechanism from a first position to a second position to release the septal occluder from the first attachment mechanism, transitioning the second locking body of the second attachment mechanism from a first position to a second position to release the septal occluder from the second locking mechanism, and retrieving the first attachment mechanism and the second attachment mechanism.

According to another embodiment, the method for delivering a medical implant to an intracardiac site in a patient includes transitioning the second attachment mechanism from a first position to a second position by rotating a second locking body of the second attachment mechanism.

In another aspect, the invention is a method for recapturing an intracardiac medical implant. The method comprises introducing a delivery system comprising a first attachment mechanism comprising a locking arm having a shaped locking portion, and a second attachment mechanism, wherein the medical implant is attached to the first attachment mechanism and the second attachment mechanism, releasing the first attachment mechanism from the implant, and recapturing the implant with the first attachment mechanism wherein the second attachment mechanism remains attached to the implant during release and recapture.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a perspective cutaway view of a heart illustrating a patent foramen ovale.

FIG. 2 is a perspective view of a delivery system, including a handle, a delivery catheter, a first locking mechanism, and a second locking mechanism, for the percutaneous transluminal closure of an intracardiac opening according to an illustrative embodiment of the invention.

FIG. 3 is a perspective view of a portion of a delivery system, including a first locking mechanism and a second locking mechanism, according to an illustrative embodiment of the invention.

FIG. 4 is a perspective view of a first locking mechanism, including a male locking member and a female lock receiving member, according to an illustrative embodiment of the invention.

FIG. 5 is a perspective view of a second locking mechanism, including a male threaded body and a female thread receiving body, according to an illustrative embodiment of the invention.

FIGS. 6A, 6B, 6C, 6D, 6E, and 6F illustrate a series of steps for implanting an intracardiac closure device from a perspective view of a portion of a delivery system according to an illustrative embodiment of the invention.

### DETAILED DESCRIPTION OF THE INVENTION

The present invention features devices, systems and related methods for closing cardiac openings, such as, for example, the patent foramen ovale, described below. Throughout the description, the terms proximal and distal refer to the position of elements relative to the operator of the exemplary delivery system 8. Proximal is that portion of the delivery system 8 closer to the operator and distal is that portion of the delivery system 8 further away from the operator.

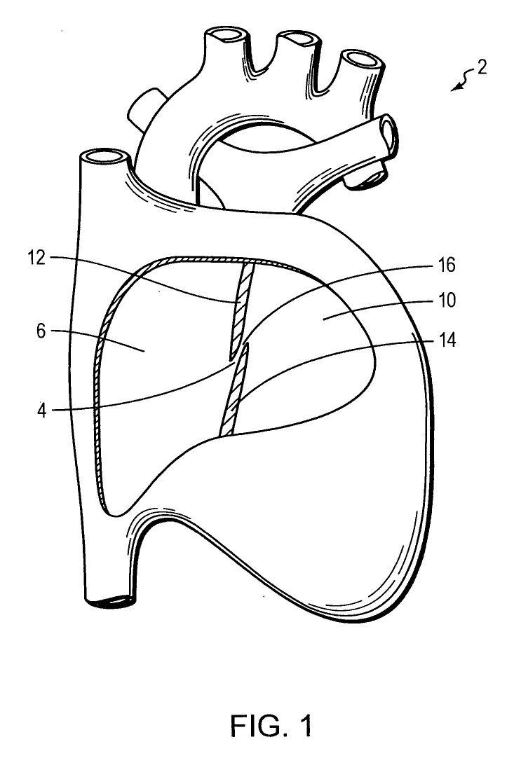

FIG. 1 depicts a cutaway view of a heart 2. The heart 2 includes a septum 4 that divides a right atrium 6 from a left atrium 10. The septum 4 includes a septum secundum 12 and a septum primum 14. An exemplary cardiac opening, a patent foramen ovale 16, that is to be corrected by the device, system and related method of the present invention is located between the septum secundum 12 and the septum primum 14. The patent foramen ovale 16 provides an undesirable fluid communication between the right atrium 6 and the left atrium 10 and, under certain conditions, allows for the shunting of blood and toxins carried by the blood between the right atrium 6 and the left atrium 10. If the patent foramen ovale 16 is not closed or obstructed in some manner, a patient is placed at higher risk for an embolic stroke in addition to other circulatory abnormalities.

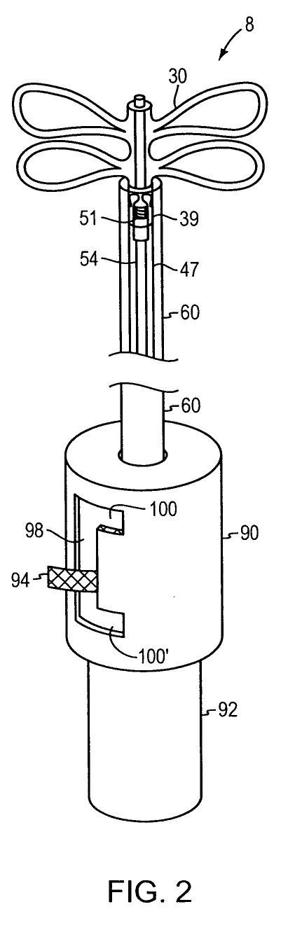

In overview, the delivery system 8 according to the invention illustrated, for example, in FIG. 2, includes a dual attachment mechanism, described in greater detail below, that allows the delivery and release of a medical implant, for example, an intracardiac septal occluder 30, with both a relatively more rigid first attachment mechanism 39 and a relatively more flexible second attachment mechanism 51. An exemplary septal occluder 30 is disclosed in co-owned patent applications U.S. Ser. No. 10/890,784 and U.S. Ser. No. 11/395,718, both entitled “Tubular Patent Foramen Ovale (PFO) Closure Device with Catch System,” herein incorporated by reference in their entirety.

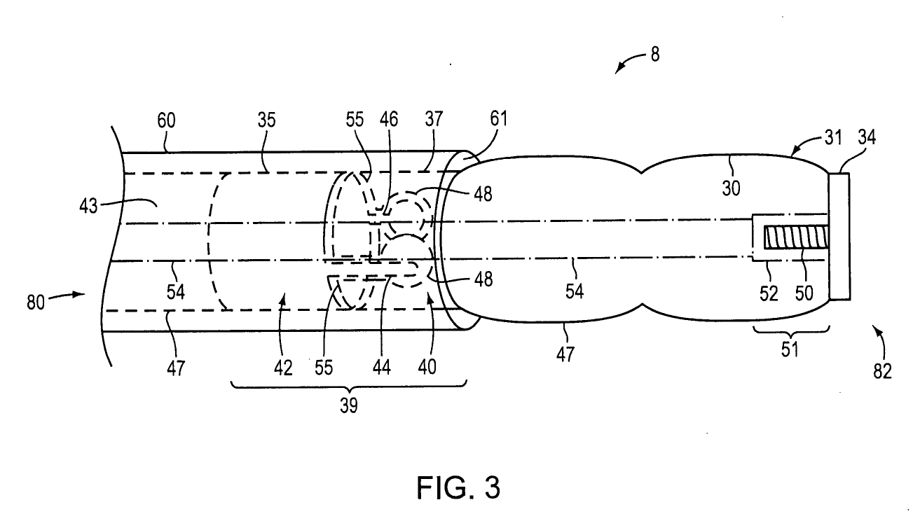

In the exemplary embodiment of the delivery system 8 illustrated in FIGS. 2 and 3, the first attachment mechanism 39 is secured to a substantially rigid first elongate member 47. The rigidity of the first elongate member 47 provides the operator with substantial control over the placement of the septal occluder 30. Following initial placement of the septal occluder 30 at the anatomical site, the first attachment mechanism 39 can be released, while maintaining the second attachment mechanism 51 in its locked state. The second attachment mechanism 51 remains secured to a relatively more flexible second elongate member 54, relative to the more rigid first elongate member 47, allowing the operator to make minor adjustments in the placement of the septal occluder 30 prior to final release of the septal occluder 30 by release (unlocking) of the second attachment mechanism 51 and removal of the delivery system 8 from the anatomical site when the septal occluder 30 is released and implanted.

FIG. 2 is a perspective view of an exemplary delivery system 8 according to an illustrative embodiment of the invention. The delivery system 8 includes a handle 90, a delivery catheter 60 including a lumen 61 (illustrated in FIG. 3), a first engagement actuator 94 secured to the first elongate member 47 and axially disposed in the lumen 61 of the delivery catheter 60 and a second engagement actuator 92 secured to the second elongate member 54 and axially disposed in the lumen 43 (illustrated in FIG. 3) of the first elongate member 47. In a further embodiment, the first elongate member 47 is axially disposed in the lumen 61 of the delivery catheter 60 and the second elongate member 54 is axially disposed in the lumen of the first elongate member 47.

With continued reference to FIG. 2, according to one embodiment, the first engagement actuator 94 is adapted to move distally and proximally in a longitudinal direction parallel the long axis of the delivery system 8. The first engagement actuator 94 moves distally to extend the first elongate member 47 and moves proximally to retract the first elongate member 47 relative to the delivery catheter 60. According to a further embodiment, the first engagement actuator 94 is also adapted to move in a clockwise direction and a counterclockwise direction through a portion of the circumference of the handle 90 defined by a slot 96 within the handle 90.

With continued reference to FIG. 2, according to one embodiment, the slot 96 includes an intermediate portion 98 extending axially a portion of the length of the handle 90 and terminates at a distal terminal portion 100 and a proximal terminal portion 100′. Each of the distal terminal portion 100 and the proximal terminal portion 100′ extend substantially perpendicular to the intermediate portion 98 of the slot 96 and extend around a portion of the circumference of the handle 90. When the first engagement member 94 is located in either the distal terminal portion 100 or the proximal terminal portion 100′, the first engagement member 94 is prevented from further longitudinally extending or retracting. With the first engagement actuator 94 locked in this position, the first engagement actuator 94 prevents the connected first elongate member 47 from moving longitudinally relative to the delivery catheter 60. According to alternative embodiments, the slot 96 includes zero (not shown), one 100, two 100, 100′, three 100, 100′, 100″ (not shown), four 100, 100′, 100″, 100′″ (not shown) or five 100, 100′, 100″, 100′″, 100″″ (not shown) terminal portions disposed along the length of the intermediate portion 98 of the slot 96.

With continued reference to FIG. 2, the first engagement actuator 94 can be maneuvered through the intermediate portion 98 of the slot 96 to extend or retract the first elongate member 47. The first engagement actuator 94 can also be maneuvered through the distal terminal portion 100 and the proximal terminal portion 100′ of the slot 96 to lock the first elongate member 47. The first elongate member 47 can be locked in either a retracted position, when the first engagement actuator 94 is located in the distal terminal portion 100, or an extended position, when the first engagement actuator 94 is located in the proximal terminal portion 100′.

With continued reference to FIG. 2, according to one embodiment, the second engagement actuator 92 is adapted to move distally and proximally in a longitudinal direction parallel the long axis of the delivery system 8 to, respectively, extend and retract the second elongate member 54 relative to the delivery catheter 60 in which it is disposed. According to one embodiment, the second engagement actuator 92 is adapted to move in a clockwise direction and a counterclockwise direction through at least a portion of the circumference of the handle 90 or through the entire circumference of the handle 90.

FIG. 3 is a perspective view of a portion of a delivery system 8, including a delivery catheter 60, a first attachment mechanism 39 and a second attachment mechanism 51, according to an illustrative embodiment of the invention. According to one embodiment of the invention, the first attachment mechanism 39 includes a first locking member, for example, a male locking member 42, connected to the distal end of and contiguous with a first elongate member 47. According to one embodiment, the first elongate member 47 further comprises an axially positioned lumen 43. The first elongate member 47, in one embodiment, extends from the first engagement actuator 94 within the handle 90, shown in FIG. 2, at the proximal end 80 of the delivery system 8 to the male locking member 42 of the first attachment mechanism 39 at the distal end 82 of the delivery system 8.

With continued reference to FIG. 3, according to one embodiment of the invention, the second attachment mechanism 51 includes a second locking body, for example, a female thread receiving body 52, connected to the distal end of and contiguous with a second elongate member 54. The second elongate member 54, in one embodiment, extends from the second engagement actuator 92 within the handle 90, shown in FIG. 2, at the proximal end 80 of the delivery system 8 to the female thread receiving body 52 of the second attachment mechanism 51 at the distal end 82 of the delivery system 8.

With continued reference to FIG. 3, the first attachment mechanism 39, according to one embodiment, further includes a female lock receiving member 40 connected to and contiguous with an implant, for example, a septal occluder 30. It is contemplated that the relative position of the male locking member 42 and the female lock receiving member 40 can be reversed, such that the female lock receiving member 40 is secured to the first elongate member 47 and the male locking member 42 is secured to an implant, for example, a septal occluder 30.

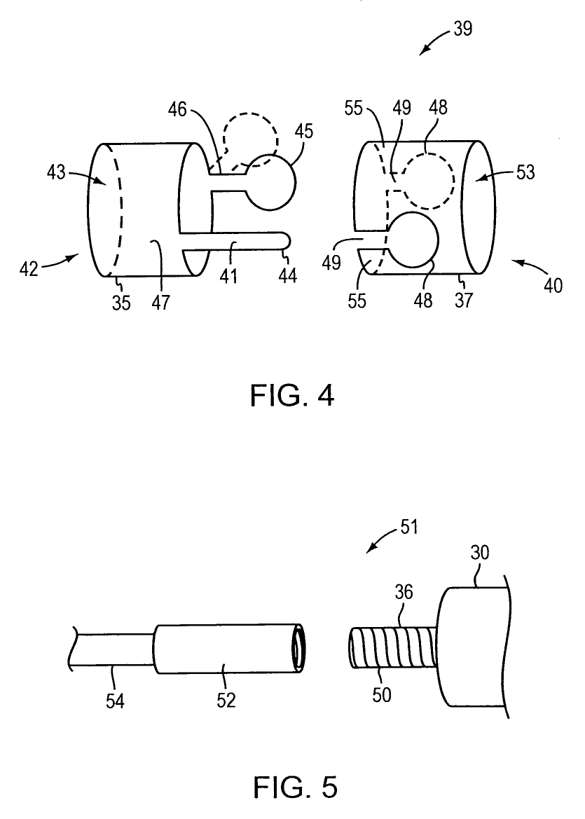

FIG. 4 is a perspective view of an exemplary first attachment mechanism 39, including the male locking member 42 and the female lock receiving member 40, according to an illustrative embodiment of the invention. In one embodiment, the male locking member 42 includes a tubular body 35 defining an open ended central lumen 43. In a particular embodiment, the second elongate member 54 and the connected second attachment mechanism 51, shown in FIG. 3, can be slideably received in the central lumen 43 of the tubular body portion 35 of the first attachment mechanism 39. According to another embodiment, the second elongate member 54 and the connected second attachment mechanism 51 can be slideably received in the lumen 61 of the delivery catheter 60 (not shown) parallel to the first elongate member 47 and connected first attachment mechanism 39.

In a particular embodiment of the invention, shown in FIG. 4, the male locking member 42 further includes one or more elongate fingers 44 projecting outward distally from a distal end of the tubular body 35. In one embodiment, the guide fingers 44 are straight, elongate pins with, for example, arcuately shaped ends. The shaped ends of the guide fingers 44 are not limited to those illustrated, but may assume other shapes as well, including, for example, rounded, elliptical, square, pointed or triangular. Although one guide finger 44 is shown in FIG. 4, more than one guide finger 44 can be provided on the male locking member 42, for example, two 44, 44′, three 44, 44′, 44″ or four 44, 44′, 44″, 44′″ guide fingers. According to a particular embodiment of the invention, the outer surface 41 of each guide finger 44 is preferably coextensive with the outer surface 41 of the tubular body 35 of the male locking member 42.

With continued reference to FIG. 4, according to one embodiment of the invention, the male locking member 42 further includes one or more elongate locking arms 46. In one embodiment, the elongate locking arm 46 includes a shaped locking portion 45 formed at a distal end of the elongate locking arm 46 which extends outward distally and angles outward radially from the distal end of the tubular body 35. In one embodiment, the exemplary locking arm 46 is substantially equal in width to the width of the guide finger 44. In other embodiments the locking arm 46 may be unequal to the width of the guide finger 44. In another embodiment, underlying each locking arm 46 is a slot (not shown) formed in the tubular body 35 to receive the locking arm 46.

With continued reference to FIG. 4, according to one embodiment of the invention, each locking arm 46 of the male locking member 42 can be formed to, for example, extend outward distally and angle outward radially beyond an outer surface 47 of the tubular body 35 of the male locking member 42, as illustrated in ghost outline in FIG. 4, when the locking arm 46 is unconfined. Although one locking arm 46 is shown in FIG. 4, more than one locking arm 46 can be provided, for example, two, three, or four locking arms 46.

Referring still to FIG. 4, a shaped locking portion 45 can be formed at the distal end of each locking arm 46 of the male locking member 42. According to a preferred embodiment, the shaped locking portion 45 extends outward laterally in a distal direction from at least one side of the locking arm 46 and, for example, forms a portion of a circle. According to alternative embodiments (not shown), the shaped locking portion 45 can be in the form of an ellipse, a T, a rectangle, a square, a hook, a triangle, or an L, for example. The shaped locking portion 45 of the male locking member 42 facilitates engagement with a lock receiving portion 48 of the female lock receiving member 40. According to one embodiment, the guide fingers 44 and the locking arms 46 can be spaced a substantially equal distance apart around the circumference of the tubular body 35 of the male locking member 42.

Referring still to FIG. 4, the guide fingers 44 and the locking arms 46, in one embodiment, can be equal in number and, although only one guide finger 44 and one locking arm 46 are shown, more than one guide finger 44 or locking arm 46 can be used, for example, two guide fingers 44, 44′ and two locking arms 46, 46′, or three guide fingers 44, 44′, 44″ and three locking arms 46, 46′, 46″. According to another embodiment, the guide fingers 44 and the locking arms 46 can be unequal in number, for example, one guide finger 44 and two locking arms 46, 46′, one guide finger 44 and three locking arms 46, 46′, 46″, two guide fingers 44, 44′ and one locking arm 46, or three guide fingers 44, 44′, 44″ and one locking arm 46.

With continued reference to FIG. 4, according to one embodiment, the female lock receiving member 40 includes a tubular body 37 which defines an open ended central lumen 53 for receiving the second attachment mechanism 51, shown in FIG. 3. In one embodiment, the tubular body 37 of the female lock receiving member 40 is substantially equal in diameter to the tubular body 35 of the male locking member 42 so that the two are coextensive when the male locking member 42 and the female lock receiving member 40 are engaged.

With continued reference to FIG. 4, the female lock receiving member 40, in one embodiment according to the invention, also includes a plurality of lock receiving portions 48 which can be shaped to conform to and to receive the guide fingers 44 and the locking arms 46 with shaped locking portions 45 of the male locking member 42. In one embodiment, the number of lock receiving portions 48 is equal to the combined number of guide fingers 44 and locking arms 46. According to another embodiment, there are more lock receiving portions 48 than the combined number of guide fingers 44 and locking arms 46.

With continued reference to FIG. 4, extending into each of the lock receiving portions 48 of the female lock receiving member 40, in one embodiment, is a straight, open ended, cutout entry channel 49 which can be formed to receive either a guide finger 44 or a locking arm 46 of the male locking member 42. In one embodiment, the lock receiving portions 48 of the female lock receiving member 40 can be spaced a substantially equal distance apart around the circumference of the tubular body 37 of the female lock receiving member 40 to conform to the spacing of the guide fingers 44 and locking arms 46 of the male locking member 42.

With continued reference to FIG. 4, according to one embodiment, outwardly projecting spacer sections 55 extend outwardly between adjacent cutout entry channels 49. In one embodiment (not shown), the spacer sections 55 terminate in an inclined outer surface which forms an apex (not shown). According to one embodiment, the inclined outer surface angles downwardly toward the cutout entry channel 49 and the combination of a first inclined outer surface of a first spacer section 55 and a second inclined outer surface of a second adjacent spacer section 55′ form an enlarged outwardly tapered opening (not shown) for each cutout entry channel 49.

Referring again to FIG. 4, according to one embodiment, the first attachment mechanism 39 is in a second open, for example, unlocked, position when each locking arm 46 of the male locking member 42 is unconfined by the delivery catheter 60, shown in FIG. 3, and angles outwardly from a longitudinal axis and beyond an outer surface of the tubular body 35 of the male locking member 42, as illustrated in ghost outline in FIG. 4.

As shown in FIG. 3, according to another embodiment, the first attachment mechanism 39 transitions from the second open position to a first closed, for example, locked, position when a delivery catheter 60 is extended from a proximally retracted position to a distally extended position to engage the male locking member 42 and female lock receiving member 40. According to another embodiment, the delivery catheter 60 also engages the tubular body 35, guide fingers 44 and locking arms 46 of the male locking member 42, and the lock receiving portions 48 and tubular body 37 of the female lock receiving member 40.

With continued reference to FIG. 3, according to one embodiment, the male locking member 42 is moved toward the female lock receiving member 40 until the guide fingers 44 of the male locking member 42 engage the tapered openings (not shown) of the cutout entry channels 49. As the male locking member 42 continues to move toward the female lock receiving member 40, each guide finger 44 is guided into the female lock receiving member 40 by the cutout entry channel 49. When the guide fingers 44 are positioned in their cutout entry channels 49, the locking arms 46 of the male locking members 42 are positioned above and in alignment with their associated cutout entry channels 49.

As the delivery catheter 60 is extended toward the distal portion 82 of the delivery system 8, the delivery catheter 60 applies pressure to the outwardly extended locking arms 46 of the male locking member 42. The pressure of the delivery catheter 60 compresses the locking arms 46 inward radially and substantially parallel to a longitudinal axis of the first attachment mechanism, for example, into the lock receiving portions 48 of the female lock receiving member 40. The guide fingers 44 and locking arms 46 of the male locking member 42, according to one embodiment, can be locked into and remain connected to the lock receiving portions 48 of the female lock receiving member 40 while the delivery catheter 60 remains in its extended closed first position.

With continued reference to FIG. 3, according to one embodiment, the delivery catheter 60 can be retracted toward the proximal portion 80 of the delivery system 8 to transition the first attachment mechanism 39 from a closed first position to an open second position. Retracting the delivery catheter 60 also reveals the tubular body 37 and lock receiving portions 48 of the female lock receiving member 40, and the guide fingers 44, locking arms 46, and tubular body 35 of the male locking member 42. According to one embodiment, as the delivery catheter 60 is retracted, the locking arms 46 of the male locking member 42 return to their radially outwardly extended open second position (as illustrated in ghost outline in FIG. 4) beyond the outer surface of the tubular body 35 of the male locking member 42.

With continued reference to FIG. 3, when the locking arms 46 are in their outwardly extended open second position, according to one embodiment, the first attachment mechanism is released, the male locking member 42 and female lock receiving member 40 are free to disconnect axially and are no longer engaged. According to an alternative embodiment, the first elongate member 47, secured to the male locking member 42, can be advanced toward the distal portion 82 of the delivery system 8. Advancing the first elongate member 47 exposes the male locking member 42 beyond the opening 61 of the delivery catheter 60 and transitions the first attachment mechanism 39 from a first closed position to an open second position. Transitioning the first elongate member 47 to an open second position releases the first attachment mechanism 39. According to another embodiment, the delivery catheter 60 reversibly and repeatedly extends distally and retracts proximally, reversibly and repeatedly transitioning between a first closed position and a second open position, to engage and release, respectively, the first attachment mechanism 39.

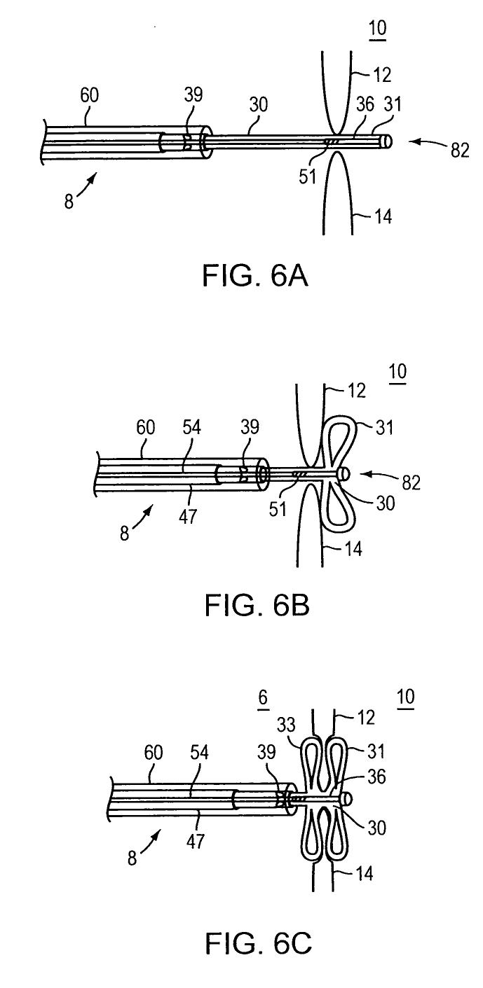

FIG. 5 is a perspective view of a portion of the second attachment mechanism 51, including a first locking body 50 and a second locking body 52, according to an illustrative embodiment of the invention. According to one embodiment, the first locking body 50 and the second locking body 52 can be a male threaded body 50 and a female thread receiving body 52. In one embodiment, the male threaded body 50 connects to a cap 34 on the distal portion 31 of the medical implant, e.g., a septal occluder 30, as illustrated in FIG. 3.

According to alternative embodiments, the second attachment mechanism 51 can be any suitable attachment mechanism. One exemplary second attachment system (not shown) comprises two members, e.g., balls, attached to a delivery string. Ball is optionally formed of the same material as the occluder and designed such that, upon the application of sufficient pulling force to delivery string, ball passes through a central tube of the occluder to lock occluder in a closed position.

According to another embodiment, the second attachment mechanism 51 comprises a delivery string attached to a third elongate member 36 of the occluder 30. Upon application of sufficient pulling force to the delivery string, the proximal end of the second elongated member 54 passes through a central tube of the occluder 30 to lock the occluder 30 in a deployed position.

Another exemplary second attachment system (not shown) comprises a hollow third elongate member 36 having at least two half-arrows located at its proximal end and attached to a delivery string. Upon the application of sufficient pulling force to delivery string, half-arrows pass through the central tube of the occluder, expand to their original shape and arrangement, and lock the occluder in a closed position.

Additional exemplary second attachment mechanisms include a threaded screw, a tie-wrap, or a combination of second attachment mechanisms. These and other second attachment mechanisms are described in greater detail in co-owned U.S. patent applications U.S. Ser. No. 10/890,784 and U.S. Ser. No. 141/395,718, the teachings of which are incorporated by reference herein in their entirety.

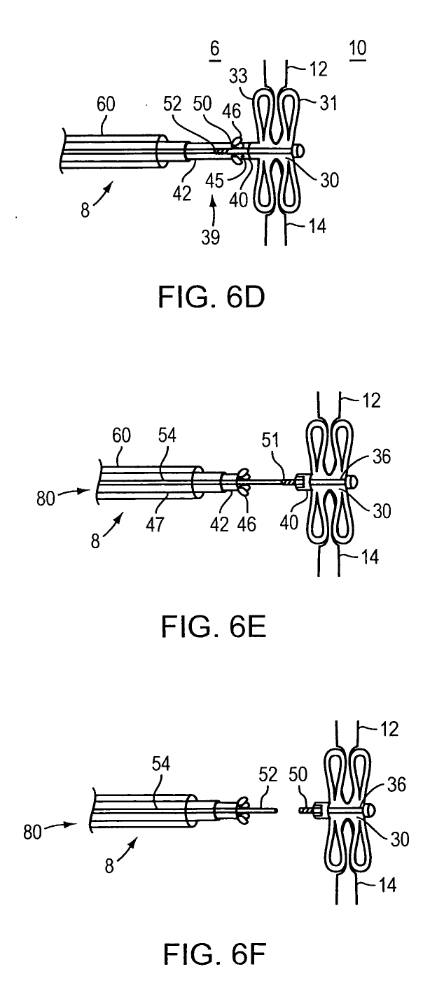

As illustrated in FIG. 6A, in another embodiment, the first attachment mechanism 39 further includes a third elongate member 36 connected to and contiguous with, on a first end, the septal occluder 30 or the cap 34 of the septal occluder 30. On a second end, the third elongate member 36 is connected to and contiguous with the male threaded body 50 or the female thread receiving body 52. The male threaded body 50 connects to the third elongate member 36 on the septal occluder 30.

The female thread receiving body 52, according to one embodiment of the invention illustrated in FIG. 5, connects to the second elongate member 54. In one embodiment of the invention, the second attachment mechanism 51 can be transitioned from a first engaged position to a second disengaged position by an operator transitioning, for example, rotating, twisting or releasing a suture from, the second elongate member 54 by its proximal portion 80 (illustrated in FIG. 6E) to release the female thread receiving body 52 from the male threaded body 50.

As illustrated in FIG. 3, in a particular embodiment, the second elongate member 54 extends axially through the lumen 43 of the first elongate member 47. Alternatively, the second elongate member 54 extends longitudinally in the lumen 61 of the delivery catheter 60 parallel to the first elongate member 47.

Referring to FIG. 5, according to one embodiment of the invention, the first locking body 50 is secured to the septal occluder 30 and the second locking body 52 is secured to the second elongate member 54, e.g., by extrusion, welding, soldering, molding, threading, or by an adhesive. According to a preferred embodiment, the first locking body 50 can be coextensive with and formed of the same material as the septal occluder 30. It is contemplated that the relative position of the male threaded body 50 and the female thread receiving body 52 can be reversed, such that the male threaded body 50 can be secured to the second elongate member 54 and the female thread receiving body 52 can be secured to the septal occluder 30.

According to alternative embodiments, the second attachment mechanism 51 can be in the form of any operator controlled, reversible attachment mechanism. According to one embodiment, the reversible second attachment mechanism is a ball and socket joint, a ball and loop joint, a ball-to-ball connection, or a pin-to-pin connection, as disclosed in U.S. Ser. No. 60/662,502. According to another embodiment, the reversible second attachment mechanism is a tensioned clamp formed of two, three, or more prongs or a lobster claw clamp, as disclosed in U.S. Ser. No. 10/389,471. According to a further embodiment, the reversible second attachment mechanism is a collet, as disclosed in U.S. Ser. No. 10/389,478. According to another embodiment, the reversible second attachment mechanism is a magnetic attraction system, as disclosed in U.S. Ser. No. 10/379,058. According to a further embodiment, the reversible second attachment mechanism is a releasable knot or suture, as disclosed in U.S. Ser. No. 10/944,512. The reversible second attachment mechanism may assume any other attachment and release mechanism known or contemplated by those of skill in the art. The teachings of each of the foregoing patent applications are herein incorporated by reference in their entirety.

FIGS. 6A, 6B, 6C, 6D, 6E, and 6F illustrate an exemplary series of steps according to one embodiment of the invention for implanting a medical implant, for example a septal occluder 30, with the delivery device 8 according to the invention described above. The septal occluder 30 deploys in a series of steps on the left atrial and right atrial sides of the intracardiac opening, for example, between the septum secundum 12 and septum primum 14 of a patent foramen ovale.

Referring to FIG. 6A, according to one embodiment, the delivery system 8 can be inserted into a patient via a percutaneous, transluminal route, e.g., the femoral vein. According to this embodiment, the septal occluder 30 can be in a collapsed configuration with both the first attachment mechanism 39 and the second attachment mechanism 51 in their locked configurations. Optionally, according to another embodiment, an introducer sheath (not shown) can be used to introduce the delivery system 8 into the body. The distal portion 82 of the delivery system 8, in one embodiment, can be inserted into the lumen of the patent foramen ovale, located between the septum secundum 12 and the septum primum 14, and into the left atrium 10. According to another embodiment, the distal portion 31 of the septal occluder 30 can be inserted into the lumen of the patent foramen ovale and into the left atrium 10.

According to one embodiment of the invention, as illustrated in FIG. 3, when the locking arm 46 is compressed by the slideable delivery catheter 60, the first attachment mechanism 39 is in a first closed position, a locked configuration. When the first attachment mechanism 39 is in the first closed position, the locking arm 46 and its shaped locking portions 45 are linear and coextensive with the tubular body 35 of the male locking member 42 and with the attached first elongate member 47.

According to another embodiment of the invention, also illustrated in FIG. 3, when the male threaded body 50 and the female thread receiving body 52 are threaded together, the second attachment mechanism 51 is in a first engaged position, a locked configuration. When in the first engagement position, the male threaded body 50 and the female thread receiving body 52 are not able to separate when linear force is applied. Exemplary linear forces include an operator induced distal push or proximal pull of the attached second elongate member 54.

Referring now to FIG. 6B, according to one embodiment, the distal portion 31 of the septal occluder 30 can be extended distally from within the lumen 61 of the delivery catheter 60 and deployed into the left atrium 10. According to one embodiment, while the first attachment mechanism 39 and the delivery catheter 60 are maintained in a stationary position, the second elongate member 54, attached to the second attachment mechanism 51, can be retracted proximally by the operator of the delivery system 8. With reference to FIG. 2, the second attachment mechanism 51, connected to the second elongate member 54, and in turn connected to the second engagement member 92, can be retracted proximally by the operator of the delivery system 8 by retracting the second engagement member 92 proximally through a length of the intermediate portion 98 of the slot 96 on the handle 90. According to another embodiment, while the second attachment mechanism 51 is maintained in a stationary position, the first attachment mechanism 39 and the delivery catheter 60 can be together extended distally by the operator of the delivery system 8. According to this embodiment, the entire delivery system 8 then can be retracted proximally until the distal portion 31 of the septal occluder 30 is adjacent the left atrial 10 surface of the septum primum 14 and the septum secundum 12. When retracted, according to this embodiment, the distal portion 31 of the septal occluder 30 compresses axially and expands radially within the left atrium 10.

Referring now to FIG. 6C, according to one embodiment, with the distal portion 31 of the septal occluder 30 radially expanded within the left atrium 10, the proximal portion 33 of the septal occluder 30 remains in the right atrium 6. According to one embodiment, the delivery catheter 60 can be retracted proximally to expose the proximal portion 33 of the septal occluder 30 within the right atrium 6. According to one embodiment, while continuing to maintain the first attachment mechanism 39 and the delivery catheter 60 in a stationary position relative to one another to prevent the first attachment mechanism 39 from disengaging, the operator advances both the first elongate member 47 and the delivery catheter 60 toward the distal portion 82 of the delivery system 8 to axially compress and radially expand the proximal portion 33 of the septal occluder 30. According to another embodiment, while maintaining the first attachment mechanism 39 and the delivery catheter 60 in a stationary position relative to one another to prevent the first attachment mechanism from disengaging, the operator retracts the second elongate member 54 proximally to axially compress and radially expand the proximal portion 33 of the septal occluder 30.

Referring now to FIG. 6D, according to one embodiment, with the radially expanded distal portion 31 of the septal occluder 30 positioned in the left atrium 10 and the radially expanded proximal portion 33 of the septal occluder 30 positioned in the right atrium 6, the delivery system 8 can be disengaged and retracted. According to one embodiment, the first attachment mechanism 39 can be disengaged before the second attachment mechanism 51 is disengaged. According to another embodiment, the second attachment mechanism 51 can be disengaged before the first attachment mechanism 39 is disengaged.

In one embodiment, the first attachment mechanism 39 can be disengaged and transitioned from a first closed position to a second open position by first axially retracting the delivery catheter 60 in a proximal direction. In another embodiment, the first attachment mechanism 39 can be disengaged and transitioned from a first closed position to a second open position by axially extending the first attachment mechanism 39 in a distal direction while maintaining the delivery catheter 60 in a stationary position relative to the first attachment mechanism 39. In one embodiment, when the delivery catheter 60 is retracted relative to the first attachment mechanism 39 to reveal the male locking member 42 and the female lock receiving member 40, the locking arm 46 of the male locking member 42 is released from the inward radial tension of the delivery catheter 60. When released, the locking arm 46 of the male locking member 42 reassumes its radially extended configuration, and releases from the shaped locking portion 45 of the female lock receiving member 40. According to this embodiment, the locking arm 46 is no longer axially aligned with the tubular body 35 of the male locking member 42, with the attached first elongate member 47, or with the tubular body 37 of the female lock receiving member 40. In this position, the first attachment mechanism 39 is disengaged.

Referring now to FIG. 6E, according to one embodiment, once the locking arm 46 of the male locking member 42 is disengaged from the female lock receiving member 40, the delivery catheter 60 and the first elongate member 47 can be further axially retracted toward the proximal portion 80 of the delivery system 8. In one embodiment, with the first attachment mechanism 39 disengaged, the operator can adjust the position of the septal occluder 30 within the intracardiac opening 12, 14 using the second elongate member 54 while the second attachment mechanism 51 remains engaged. Once the operator is satisfied with the position of the septal occluder 30, the second attachment mechanism 51 can be disengaged and retracted.

Referring now to FIG. 6F, according to one embodiment, with the septal occluder 30 properly positioned, the operator can disengage and retract the second attachment mechanism 51. According to one embodiment of the invention, the second elongate member 54 and the attached female thread receiving body 52 can be transitioned from a first engaged position to a second disengaged position by the operator. According to one embodiment, the second attachment mechanism 51 is disengaged by rotating the second engagement member 92 on the handle 90 of the delivery system 8, illustrated in FIG. 2 to disengage the female thread receiving body 52 from the male threaded body 50. According to another embodiment, the second attachment mechanism 51 is disengaged by twisting the second engagement member 92 to disengage, for example, a ball from a socket joint or a ball from a loop joint. According to a further embodiment, the second attachment member 51 is disengaged by grasping the second engagement member 92 to disengage the second attachment mechanism 51, for example, a collet or lobster claw clamp. According to another embodiment, the second attachment member 51 is disengaged by releasing or removing a suture. According to each of these embodiments, disengaging the second attachment mechanism 51 releases the septal occluder 30 from the delivery system 8. In one embodiment, the second elongate member 54 and the attached female thread receiving body 50 then can be retracted longitudinally toward the proximal portion 80 of the delivery system 8 by the operator. The septal occluder 30 remains positioned on the left atrial and right atrial sides of the septum primum 14 and the septum secundum 12 of the patent foramen ovale.

According to another illustrative embodiment of the invention, at some time following deployment of both the distal portion 31 and the proximal portion 33 of the septal occluder 30, but before release of both the first attachment mechanism 39 and the second attachment mechanism 51, the septal occluder 30 can be recaptured from and/or repositioned within the intracardiac opening using the delivery system 8. According to one embodiment, the septal occluder 30 is partially deployed when the distal portion 31 of the septal occluder 30 is deployed in the left atrium 10 and both the first attachment mechanism 39 and the second attachment mechanism 51 remain engaged. According to another embodiment, the septal occluder 30 is partially deployed when the distal portion 31 of the septal occluder 30 is deployed in the left atrium 10 and the proximal portion 33 of the septal occluder 30 is deployed in the right atrium 6 and both the first attachment mechanism 39 and the second attachment mechanism 51 remain engaged. In one embodiment, the septal occluder 30 can be recaptured from between the left atrial and right atrial sides of the intracardiac opening by extending the second elongate member 54 toward the distal end 82 of the delivery device 8 to collapse the distal portion 31 and/or the proximal portion 33 of the septal occluder 30. In another embodiment, the distal portion 31 and/or the proximal portion 33 of the septal occluder 30 can be collapsed by retracting the first elongate member 47 toward the proximal end 80 of the delivery system 8. According to one embodiment, the collapsed septal occluder 30 can be repositioned and redeployed between the left atrial and right atrial sides of the intracardiac opening. According to another embodiment, the delivery device 30 and the collapsed septal occluder 30 can be removed from the intracardiac opening, optionally further removed from the patient.

According to another illustrative embodiment of the invention, at some time following full deployment of the septal occluder 30, the septal occluder 30 can be recaptured from and/or repositioned within the intracardiac opening using the delivery system 8. In one embodiment, the male locking member 42 of the first attachment mechanism 39 is disengaged from the female lock receiving member 40 and the second attachment mechanism 51 remains engaged. According to this embodiment, the male locking member 42 of the first attachment mechanism 39 can be reengaged with the female lock receiving member 40 of the first attachment mechanism 39 on the septal occluder 30. The first attachment mechanism 39 then can be manipulated as previously described to recapture, reposition and/or remove the septal occluder 30 within the intracardiac opening.

In another embodiment, the female thread receiving body 52 of the second attachment mechanism 51 is disengaged from the male threaded body 50 while the first attachment mechanism 39 remains engaged. In this position, the female thread receiving body 52 of the second attachment mechanism 51 can be reengaged with the male thread receiving body 50 of the second attachment mechanism 51 on the septal occluder 30. The second attachment mechanism 51 then can be manipulated as previously described to recapture, reposition and/or remove the septal occluder 30 within the intracardiac opening.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are embraced therein.

What we claim is:

1. A delivery system for delivering a medical implant to an anatomical site in a patient, comprising:
a first attachment mechanism sized and shaped for insertion into a mammalian blood vessel, the first attachment mechanism defining a lumen and comprising a first locking member at a distal end, the first locking member comprising a locking arm having a shaped locking portion at a distal end; and
a second attachment mechanism slidably receivable within the lumen of the first attachment mechanism and comprising a second locking member at a distal end;
wherein the first attachment mechanism and the second attachment mechanism hold the medical implant for delivery to an anatomical site in a patient.

2. The delivery system of claim 1 further comprising a delivery catheter slideably disposed over the first attachment mechanism allowing the first locking member to transition between a first position and a second position, wherein the catheter immobilizes the first locking member in the first position when slid over the first locking member and releases the first locking member into the second position when slid away from the first locking member.

3. The delivery system of claim 1 wherein the first attachment mechanism reversibly transitions between a first position and a second position.

4. The delivery system of claim 3 wherein a portion of the first locking member transitions between a first position, wherein the first locking member is substantially parallel to a longitudinal axis of the first attachment mechanism, and a second position, wherein the first locking member is angled outward from a longitudinal axis of the first attachment mechanism.

5. The delivery system of claim 1 wherein the first attachment mechanism comprises a male locking member and a female lock receiving member.

6. The delivery system of claim 5 wherein the male locking member comprises the first locking member.

7. The delivery system of claim 6 wherein the male locking member further comprises a guide finger.

8. The delivery system of claim 7 wherein the first attachment mechanism comprises a tubular body co-extensive with the guide finger.

9. The delivery system of claim 1 wherein the first locking member comprises an elongate locking arm.

10. The delivery system of claim 9 wherein the elongate locking arm comprises a locking portion at a free end of the elongate locking arm.

11. The delivery system of claim 10 wherein the locking portion comprises a shape selected from the group consisting of a circle, oval, ellipse, rectangle, square, hook, triangle, L, and T.

12. The delivery system of claim 1 wherein the first attachment mechanism comprises a plurality of first locking members.

13. The delivery system of claim 12 wherein the plurality of first locking members are spaced a substantially equal distance apart around the circumference of the first locking mechanism.

14. The delivery system of claim 1 wherein the second attachment mechanism is slideably disposed within the lumen of the first attachment mechanism.

15. The delivery system of claim 1 wherein the second attachment mechanism comprises a threaded attachment member.

16. The delivery system of claim 1 wherein the second attachment mechanism comprises a ball and socket attachment member.

17. The delivery system of claim 1 wherein the second attachment mechanism comprises a tensioned clamp and ball attachment member.

18. The delivery system of claim 1 wherein the second attachment mechanism comprises a collet and ball attachment member.

19. The delivery system of claim 1 wherein the second attachment mechanism comprises a magnetic attachment member.

20. The delivery system of claim 1 wherein the second attachment mechanism comprises a releasable suture.

21. The delivery system of claim 1 further comprising a delivery sheath.

22. A method for delivering a septal occluder to an intracardiac site in a patient comprising:
positioning a delivery system comprising a first attachment mechanism and a second attachment mechanism for delivering a medical implant, wherein the first attachment mechanism comprises a first locking member, the first locking member comprising a locking arm having a shaped locking portion, and the second attachment mechanism comprises a second locking member;
positioning the septal occluder at the anatomical site;
transitioning the first attachment mechanism from a first position to a second position to release the septal occluder from the first attachment mechanism; and
transitioning the second attachment mechanism from a first position to a second position to release the septal occluder from the second attachment mechanism.

23. The method of claim 22 wherein the second attachment mechanism transitions from a first position to a second position by rotating a second locking body of the second attachment mechanism.

24. A method for recapturing an intracardiac medical implant, comprising:
introducing a delivery system comprising a first attachment mechanism and a second attachment mechanism, wherein the first attachment mechanism comprises a first locking member having a locking arm with a shaped locking portion at a distal end, and wherein the medical implant is attached to the first attachment mechanism and the second attachment mechanism;
releasing the first attachment mechanism from the implant; and
recapturing the implant with the first attachment mechanism;
wherein the second attachment mechanism remains attached to the implant during release and recapture.
